(19) United States
(12) Patent Application Publication
(10) Pub. No.: US 2008/0064034 A1
(43) Pub. Date: Mar. 13, 2008

(54) Multi-Cellular Test Systems

(75) Inventors: Charli Kruse, Herrnburg (DE); Günter Fuhr, Berlin (DE)

Correspondence Address: PEARL COHEN ZEDEK LATZER, LLP, 1500 BROADWAY 12TH FLOOR, NEW YORK, NY, 10036, US

(73) Assignee: FRAUNHOFER-GESELLSCHAFT ZUR FÖRDERUNG DER ANGEWAND, Munich (DE)

(21) Appl. No.: 11/597,167

(22) PCT Filed: Mar. 2, 2005
(86) PCT No.: PCT/EP05/02197

§ 371 Date: Nov. 21, 2006

(30) Foreign Application Priority Data
May 21, 2004 (DE) 10 2004 025 080.4

(51) Int. Cl. G01N 33/00; C12M 1/00; C12Q 1/02; G01N 33/53; C12Q 1/68

(52) U.S. Cl. 435/6; 435/287.1; 435/287.2; 435/29; 435/7.1; 435/7.7; 435/7.92

(57) ABSTRACT

The invention relates to methods for testing substances using multicellular in vitro test systems, in particular systems that resemble organs, and to devices and kits for carrying out said methods.

The invention relates to methods for testing substances using multicellular in vitro test systems, in particular systems that resemble organs, and to devices and kits for carrying out said methods.

The conducting of animal tests for pharmaceutical and cosmetic studies represents an enormous cost factor and is frequently ethically problematic. They are even completely forbidden in many countries, e.g., in Europe, for the cosmetic industry. The development of multicellular, especially human in vitro test systems represents a good alternative for many studies and such human test systems can even resemble natural human tissues/organs in their properties more closely than animal models.

In the state of the art tissue cultures from explanted tissue samples (see, e.g., U.S. Pat. No. 5,726,009 and US 2002/192638) as well as cultures of differentiated cells obtained from stem cells have previously been used. The first approach has the disadvantage that it is difficult to maintain the cultures for rather long time periods and produce rather large amounts of cells without altering the properties of the cells and/or of the tissue. In the case of the second approach the stem cells are traditionally caused to differentiate into individual cell types, e.g., nerve cells, fibroblasts, etc. by the addition of specific factors, and then the effects of various chemicals, e.g., active drug substances, on these specific cells are examined. In order to examine a broad spectrum of differentiated cells, different specific cell cultures have to be prepared and tested. When using traditional adult stem cells with a limited differentiation spectrum this usually requires the cultivation of differentiated cell cultures from different starting stem cells, often with very different requirements on the growth conditions. This is associated as a rule with more time and greater cost. This disadvantage can be partially avoided by using pluripotent embryonic stem cells that can differentiate into very different cell types of all three germ layers. However, the use of human embryonic stem cells is also questionable for ethical reasons and their availability is limited. Moreover, several different and spatially separated cell cultures are required even when using pluripotent embryonic stem cells in these traditional test systems. A further significant disadvantage of these cellular monocultures is that the action of a substance on a compound of different cells as is present in a natural tissue or organ cannot be examined.

The invention therefore has the object of providing improved multicellular, in particular human in vitro test systems and methods with which the action of substances on different cell types and in particular on a compound of different cell types as is present in natural tissues and organs can be determined in a rapid and simple manner.

The present invention is based on the finding that multipotent or pluripotent adult stem cells like those that can be obtained from exocrine glandular tissue (PCT 2004/003810) can be made to aggregate and differentiate with simple means into three-dimensional cell aggregates, so-called organoid bodies, that already contain a spectrum of at least two cell types without the addition of special differentiation factors. These organoid bodies constantly continue to grow, given a sufficient supply of nutrients, and develop tissue-like or organ-like structures in which stage they are also referred to as tissue bodies. If these organoid bodies are exposed to chemical substances their action, if present, can be determined by a morphological change or otherwise detectable change in these organoid bodies and/or in the cell types contained in them. In this manner different cell types can be rapidly tested simultaneously or successively and in particular even cell aggregates that are similar to tissue or organs can be examined.

Thus, the above-cited objects are achieved in accordance with the invention by providing a method for testing substances according to claim 1 as well as devices and kits for carrying out this method according to claims 23, 24 and 28. Advantageous embodiments of the invention constitute subject matter of the dependent claims.

In order to form the organoid bodies used in accordance with the invention multipotent or pluripotent adult stem cells are used. These pluripotent stem cells are preferably isolated from exocrine glandular tissue.

The exocrine glandular tissue can stem from an adult individual or a juvenile individual. The concept “adult” as it is used in the present application therefore refers to the development stage of the starting tissue and not to that of the donor from whom the tissue stems. “Adult” stem cells are non-embryonic stem cells.

The exocrine glandular tissue is preferably isolated from a salivary gland, lachrymal gland, sebaceous gland, sweat gland, from glands of the genital tract including the prostate, or from gastro-intestinal tissue including the pancreas, or secretory tissue of the liver. In a very preferred embodiment acinar tissue is concerned. The acinar tissue stems especially preferably from the pancreas, the parotid gland or the submandibular gland.

The adult stem cells obtained from such sources can be easily isolated and maintained in a largely non-differentiated state in a stable long-time culture without a feeder cell layer or special additives. The concept feeder cells as it is used herein comprises all cells that promote the growth of the cells that are actually to be cultivated in that they release growth factors and/or provide an extracellular matrix or prevent the differentiation of the stem cell culture.

These adult stem cells can be stimulated to differentiate without the addition of special growth- or differentiation factors in a simple manner in that they are cultivated under spatial conditions that ensure a three-dimensional contact of the cells. In a preferred embodiment these conditions are the cultivation in hanging drops such as has already been described for embryonic stem cells (Wobus et al., Biomed. Biochim. Acta 47:965-973 (1998). This method will be described in more detail in the following in the examples. It is understood, however, that alternative cultivating methods that ensure the desired three-dimensional contact of the cells and are known and available to those skilled in the art can also be used. Examples of such alternative methods are the cultivation in a moved suspension culture, the cultivation in an electromagnetic field cage or laser tweezer, the cultivation on surfaces to which the cells do not adhere or adhere only poorly, or the spreading of non-resuspended cells of the primary culture. Such surfaces can be, e.g., glass, polystyrene or surfaces treated with an anti-adhesion layer, e.g., surfaces coated with PTFE or poly-HEMA.

Under these conditions three-dimensional cell compounds or cell aggregates spontaneously develop that have been referred to as “organoid bodies” in analogy with “embryoid bodies” already described for embryonic stem cells. These organoid bodies can be transferred into suspension cultures or adhesion cultures and further cultivated. Given a sufficient supply of nutrients, these organoid bodies continue to grow and can achieve diameters of a few millimeters or more. These large organoid bodies exhibit a tissue-like structure and are also referred to in this stage as “tissue bodies” in order to distinguish them from the simple cell aggregates.

If the organoid bodies are brought back into surface culture a cellular monolayer is produced from out-growing individual cells from which monolayer multi-layer areas arise from which secondary organoid bodies are spontaneously formed with comparable properties as those of the primary organoid bodies. The organoid bodies in accordance with the invention can be stored frozen, e.g., at the temperature of liquid nitrogen, without losing their viability and their ability to reproduce, grow and differentiate.

The organoid bodies contain different cell types of all three germ layers. No differentiation factors are necessary for differentiation and the cells also do not have to be transplanted in order to differentiate. However, it can be advantageous to use such differentiation factors in order to purposefully produce larger amounts of a certain cell type or in order to generate organoid bodies with a certain cell type composition.

Differentiation factors are known in the state of the art and comprise in general, e.g., bFGF (basic fibroblast growth factor) for an increased formation of cardiac cells and fibroblasts, VEGF (vascular endothelial growth factor), DMSO and isoproterenol, fibroblast growth factor 4 (FGF4), hepatocyte growth factor (HGF) for an increased formation of cardiac and liver cells, TGF beta1 (transforming growth factor beta1) for an increased formation of cardiac cells, EGF (epidermal growth factor) for an increased formation of skin- and cardiac cells, KGF (keratinocyte growth factor) (sometimes together with cortisone) for the formation of keratinocytes, retinoic acid for an increased formation of nerve-, cardiac and kidney cells, beta-NGF (beta nerve growth factor) for an increased formation of brain-, liver-, pancreatic and kidney cells, BMP-4 (bone morphogenic protein 4) and activin-A for the formation of mesodermal cells, but are not limited to them.

Differentiated cells that can be included in the organoid bodies comprise bone cells (osteoblasts and osteoclasts), chondrocytes, adipocytes, fibroblasts (e.g. skin and tendon fibroblasts), muscle cells, endothelial cells, epithelial cells, hematopoietic cells, sensory cells, endocrine and exocrine glandular cells, glia cells, neuronal cells, oligodendrocytes, blood cells, intestinal cells, cardiac-, lung-, liver-, kidney- or pancreatic cells, but are not limited to them.

In the test method in accordance with the invention the substance to be tested is brought in contact with the organoid bodies and its effect, if present, determined by a morphological or some other detectable change in these organoid bodies or in the cell types contained in them. This test method concerns, e.g., a method for analyzing the effect of known or potential active substances or toxic substances or mutagens on all or specific cell types of the organoid bodies. A more specific embodiment concerns a method for screening active drug substances or cosmetics.

The test substance can be of a very different chemical nature, e.g., a protein, lipid, a nucleic acid, e.g., RNA, DNA or a derivative thereof, a low-molecular weight or high-molecular weight chemical compound, a chemical element or a mixture of these substances. Two or more test substances can also be tested successively or simultaneously with the same organoid bodies in order to determine, e.g., interactions of the test substances.

The contacting of the test substance(s) with the organoid bodies can take place in any suitable manner depending on of the type of test substance. Suitable methods are known to those skilled in the art. If the test substance is a nucleic acid, e.g., RNA, DNA or a derivative thereof, it can be introduced into the cells of the organoid bodies with any known method of genetic engineering including the use of vectors, viruses, electroporation, etc. A test substance that is soluble or solubilizable in the culture medium is preferably simply added to the cell culture medium in which the organoid bodies are present and the organoids are incubated with the test substance in suitable concentrations for different desired time periods. If desired, used cell culture medium may be replaced during the incubation by fresh medium with the desired concentration of test substance, e.g., in order to maintain the concentration of active substance in the medium approximately constant. Depending on the type of the active substance the effective concentrations of the test substance may vary to a great extent but can be readily determined by those skilled in the art by routine tests. The contact time may vary from a few minutes to a few hours and to several days and weeks. Contact times of several weeks are not unusual. Suitable contact times also depend on the type of the active substance and can be determined by those skilled in the art by routine tests. The treated organoids and a control without test substance that was incubated just as long are subjected to a detection method.

The detection method will depend on the type of the active substance and on the type of the change to be observed in the organoid bodies and in the differentiated cells contained in them.

A number of methods for detecting the effect of drugs or toxic substances on mammalian cells is described by A. Vickers in “In vitro Methods in Pharmaceutical Research”, Academic Press, 1997. Fundamental methods for the screening of active drug substances are described by Smith, C. G., “The Process of New Drug Development”, CRC Press, 1992, and in “Advances in Drug Discovery Techniques”, editor Alan L. Harvey, John Wiley & Sons, 1998.

In specific embodiments of the present invention the detection of the effect of a substance comprises the use of one or several methods selected from the group of protein assays, immunoassays, enzymatic assays, receptor binding assays, ELISA assays, RIA assays, electrophoretic and chromatographic assays, including HPLC, Northern blots, Southern blots, Western blots, colorimetric assays, immunohistochemical, electrophysiological methods (that is, measurements of current, voltage and impedance), microscopic and spectroscopic detection methods.

The effect of the substance may be, e.g., a change in the morphology or in the capacity for proliferation, capacity for growth or in the viability of all cell types or of specific cell types of the organoid bodies. In this instance, e.g., direct visual and optical detection methods including cytometric, microscopic and calorimetric methods can be used with advantage.

Alternatively or simultaneously, the effect may be a change in the activity of specific or of all cell types of the organoid bodies. For example, this activity change can be expressed in an increased or reduced or first-time occurrence of a detectable marker substance in or on the cells concerned. In this instance the detection of the effect of the chemical substance preferably takes place via the detection of the presence or absence or the amount of a marker substance produced by specific or all cell types of the organoid bodies.

This marker substance may be, e.g., a protein including but not limited to antibody, receptor, enzyme, hormone, ion channel, neurotransmitter, surface marker, RNA, DNA, a proteoglycan, a lectin or another suitable substance. A few cell-type-specific examples of marker substances are mentioned in the following but are in no way to be construed as limiting: PGP 9.5 and NF for nerve cells, S 100 and GFAP for glia cells, SMA for muscle cells (or myofibroblasts), type II collagen for cartilage cells, amylase and trypsin for exocrine glandular cells, insulin for endocrine glandular cells, vigilin for strongly translating cells and cytokeratin for epidermal cells, type II collagen for chondrocytes, osteonectin for osteoblasts and precursor cells, osteocalcin for mature osteoblasts, CD45, CD34, DC13 for hematopoietic cells, cTNI (cardiac troponin I), cTNT (cardiac troponin T) and ANF (atrial natriuretic factor) for cardiomyocytes, collagenase 1 and TIMP-1 (tissue inhibitor of metalloproteinase I) for fibroblasts, skeletal alpha actin and tropomyosin for striated muscle cells, lipoprotein lipase (LPL) for adipocytes, alpha fetoprotein for liver cells. A very large number of such marker substances is known in the state of the art.

These markers substances can be detected, e.g., by binding to a specific binding partner that is conjugated with a detectable group. The detectable group may be, e.g., a dye, fluorescent dye, a radioactive marking, enzyme marking, luminescence marking, magnetic resonance marking or another marking known in the state of the art. If the marker substance is a protein the binding partner will preferably be a marked or markable antibody. Such marked antibodies are already known for a plurality of marker substances and can either be acquired in trade or readily produced according to known methods. Optionally marked or markable secondary antibodies may also be used. The specific binding partner can also be unmarked but bound to an affinity column or another carrier. In these instances cells comprising the marker substances on their surface can be detected by the specific binding to these carriers.

In a few instances the marker substance can also be detected by its own activity, e.g., if an ion channel or neurotransmitter is concerned or an enzyme that can be reacted with a detectable substrate. If the marker substance is a DNA or RNA it can either be detected directly by complementary and optionally marked probes or indirectly by detecting a gene product if a complete coding sequence or a regulatory sequence is concerned. Another possibility is an increased DNA- or RNA synthesis itself or an increased DNA repair activity. Such activities can be determined, e.g., by the inclusion of nucleotides that are marked radioactively or in some other manner.

The detection may be effected while maintaining the cell structure, e.g., in microscopic and immunohistochemical methods, or with destruction of the cell structure, e.g., in an electrophoresis method such as Southern blots, Northern blots or Western blots.

In an advanced stage of the differentiation the organoid bodies have structures that are similar to tissues or organs, which are formed from two or more different cell types (cf. FIG. 2-a). Such structures are, e.g., neuromuscular structures or glia-nerve cell structures or skin cell structures. The formation of desired structures can be promoted by cultivation of the organoid bodies in a medium with special differentiation factors.

In an embodiment of the method in accordance with the invention the test substance brings about a change in these structures. This change can be, e.g., a destruction of the structures, inhibition or stimulation of the development of the structures or a change in the activity of one or more cell types of which these structures are composed. Accordingly, the detection of the effect of the test substance can consist in a detection of the change in these structures. Suitable methods of detection can comprise, e.g., a direct observation of the morphological changes optionally coupled with immunological, immunohistochemical or other detection methods.

Since, as already mentioned above, the cell type composition of the organoid bodies can be determined by cultivating them in the presence of specific differentiation factors, this means that the cell type composition of the organoid bodies can be coordinated in more specific embodiments with the supposed cell-type-specific action of the test substance. This means concretely that, e.g., organoid bodies with a high component of nerve cells or organoid bodies having primarily or exclusively neuromuscular structures or nerve-glia cell structures are used in a test system in which test substances with a supposed effect on the nervous system are examined. In analogy thereto, organoid bodies with a high component of epithelial cells or with structures that are similar to skin can be used in the testing of test substances that are supposed to act via the surface. Further such specific test systems are readily apparent to and can be realized by those skilled in the art.

In further, more specific embodiments the organoid bodies are located in hollow spaces, matrices or other carrier- and/or shaping systems. The introduction of the organoids can take place, e.g., by allowing them to grow in. The hollow spaces may be, e.g., microchannels or capillaries for measuring impedance. The detection of the effect of the test substance can then consist, e.g., in an impedance measuring. Alternatively, a gradient (e.g., pH, electrochemical, signal factors, etc.) can also be produced via an organoid body and the effect of the test substance indicated by a change in this gradient.

A further aspect of the invention relates to a device for carrying out the method in accordance with the invention and comprising the organoid bodies in a suitable container, carrier- or shaping system, means for contacting the organoid bodies with a chemical substance to be tested as well as means for the detection of a morphological or other change in these organoid bodies or in cell types contained in them. In a more specific embodiment of this aspect the organoid bodies are present in hollow spaces, e.g., microconduits or capillaries or in matrices.

In one embodiment of the invention the means for carrying out the method in accordance with the invention are provided in the form of a kit. Such a kit comprises adult stem cells or the organoid bodies or differentiated cells derived from them in a suitable culture medium for maintaining the cells or the organoid bodies, respectively, optionally cryopreserved. Furthermore, the kit may contain other auxiliary means, e.g., reagents for the cultivation and differentiation of the stem cells to organoid bodies of a desired cell-type composition, means for bringing the organoid bodies in contact with a chemical substance to be tested, means for demonstrating a morphological or other change in these organoid bodies or in the cell types contained in them.

### DESCRIPTION OF THE FIGURES

FIG. 1 schematically shows the cultivation of the stem cells in surface culture and in hanging drops as well as the formation and further cultivation of organoid bodies.

FIG. 2 shows the expression of markers of neuronal cells, glial cells and smooth muscle cells as well as of amylase and insulin in the differentiated cells of the organoid bodies.
a,b: PGP 9.5-marked nerve cells show multipolar processes that exhibit numerous varicosities. c,d: the neurofilament system (bright arrows, marked green in the original photo) extends through the pericaryon into the cytoplasmatic processes. GFAP-immunoreactive glia cells (dark arrows, marked red in the original photo) are in close vicinity. e, f: α-SMA-marked cells (dark arrows, red in the original) and NF-marked nerve cells (bright arrows, green in the original) form a primitive neuromuscular network (e), with contacts being established over considerably long distances (f). g: Immunostaining of GFAP (dark arrows, red in the original) and NF (bright arrows, green in the original) in 3 weeks old OB with concentrations of nerve- and glia cells. h: Immunostaining of α-SMA (dark arrows, red in the original) and NF (bright arrows, green in the original) in 3 weeks old OBs in an advanced stage of the formation of a neuromuscular network. i, j: Cells immunoreactive for NF were found in cross sections of 8 weeks old OBs in the direct vicinity of cells that were immunoreactive for α-SMA, similarly as in native tissues. k: A subset of cells shows a positive staining for amylase (bright arrows, green in the original). l: Another cellular subset contains granular vesicles with immunoreactivity for insulin. The nuclei are counterstained with DAPI (blue in the original).

FIG. 3 shows the expression of extracellular matrix components and cytokeratins.
a,b: Globular (a) and fibrillary (b) depots of proteoglycans yielded a staining with Alcian blue. c-e: The globular (c) and fibrillary (d) areas are immunoreactive for the cartilage matrix protein collagen II. Two individual cells (e) show a cytoplasmatic marking of collagen II. f: Cells that are immunoreactive for cytokeratins are arranged in clusters. g: Confocal laser scanning microscopy of an OB. The collagen II immunoreactivity (dark arrows, marked red in the original) increases toward the middle of the OB. Vigilin-immunoreactive cells (bright arrows, marked green in the original) are localized primarily on the outer edge of the OB, which indicates their high translational activity. The nuclei are counterstained with DAPI.

FIG. 4 shows the transmission electron microscopy of differentiated OB.
a-c: smooth muscle cells with myofilaments. The myofilament system extends throughout the cytoplasm in disseminated bundles (a) and displays typical dense bodies (arrows) (b). The myoblasts display stellate cellular processes that form a connecting network (c). d: cellular processes with an accumulation of numerous small-size vesicles that most likely correspond to nerve fiber varicosities. e: Collagen- and reticular fibers. f-h: Secretory cells display electrodense vesicles. f). Secretory cells frequently contact each other in order to form acinus-like structures (g). A subset of secretory cells contains vesicles (arrow) corresponding to ultrastructural features of endocrine granulae (h), e.g., beta granulae of insulin-producing cells. l: Beginning of formation of an epithelial surface (arrow) in eight weeks old OBs. j: Typical cell contacts between keratinocytes and desmosomes (arrows).

FIG. 5A shows comparative micrographs of an untreated organoid body and of an organoid body treated with puromycin.

FIG. 5B shows Western blots of protein separations of the homogenates of the organoids shown in FIG. 5A with the detection of the translation marker vigilin.

FIG. 6 shows Western blots of protein separations of the homogenates of organoid bodies treated with and not treated with retinoic acid with the detection of the protein marker α-SMA (FIG. 6A) or the detection of neurofilaments (NF) (FIG. 6B), respectively.

FIG. 7 shows Western blots of protein separations of the homogenates of organoid bodies treated with and not treated with HGFR with the detection of the liver protein α-fetoprotein.

FIG. 8 shows Western blots of protein separations of the homogenates of organoid bodies treated with and not treated with conditioned medium of chondrocyte primary cultures with the detection of the cartilage protein collagen II.

FIG. 9 shows a basic structure and test course for the testing of substances using the methods and systems in accordance with the invention.

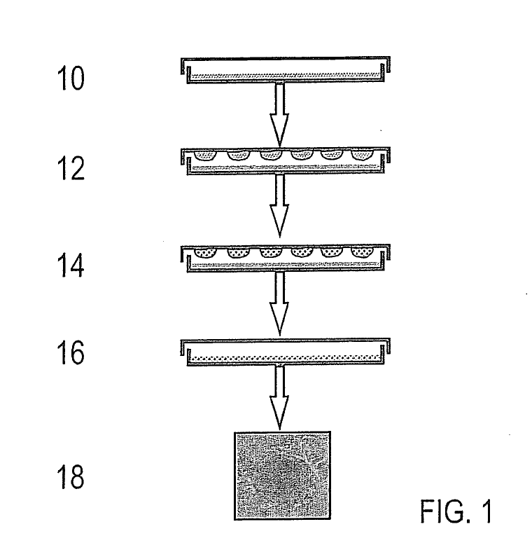

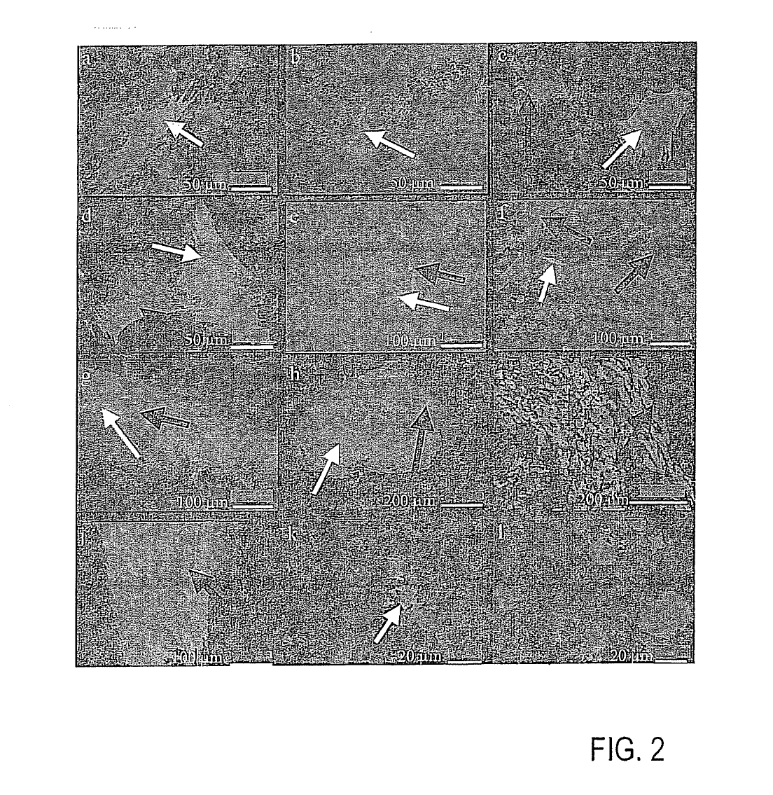

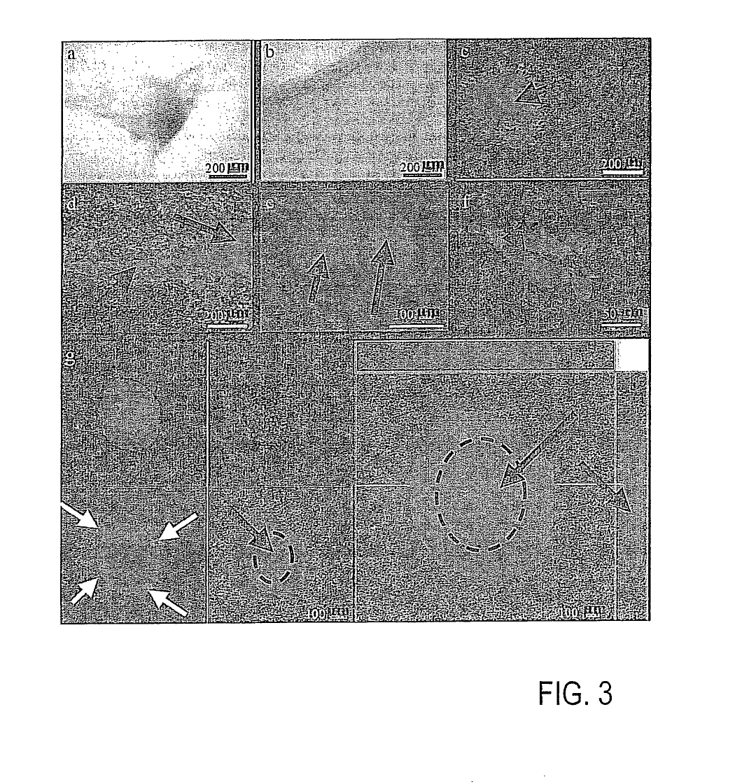

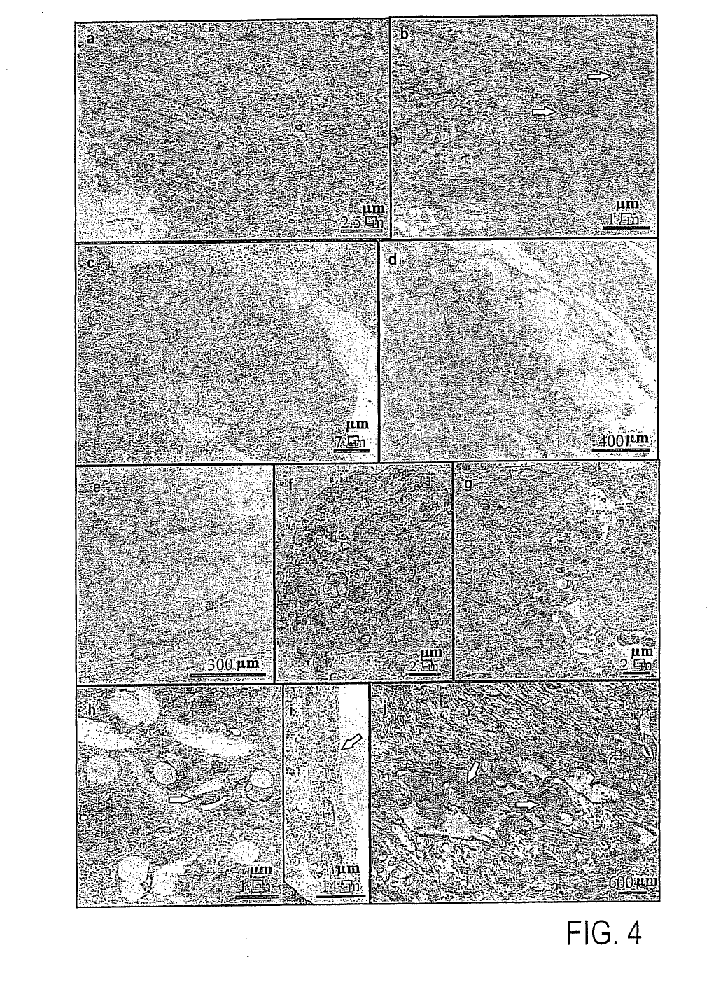

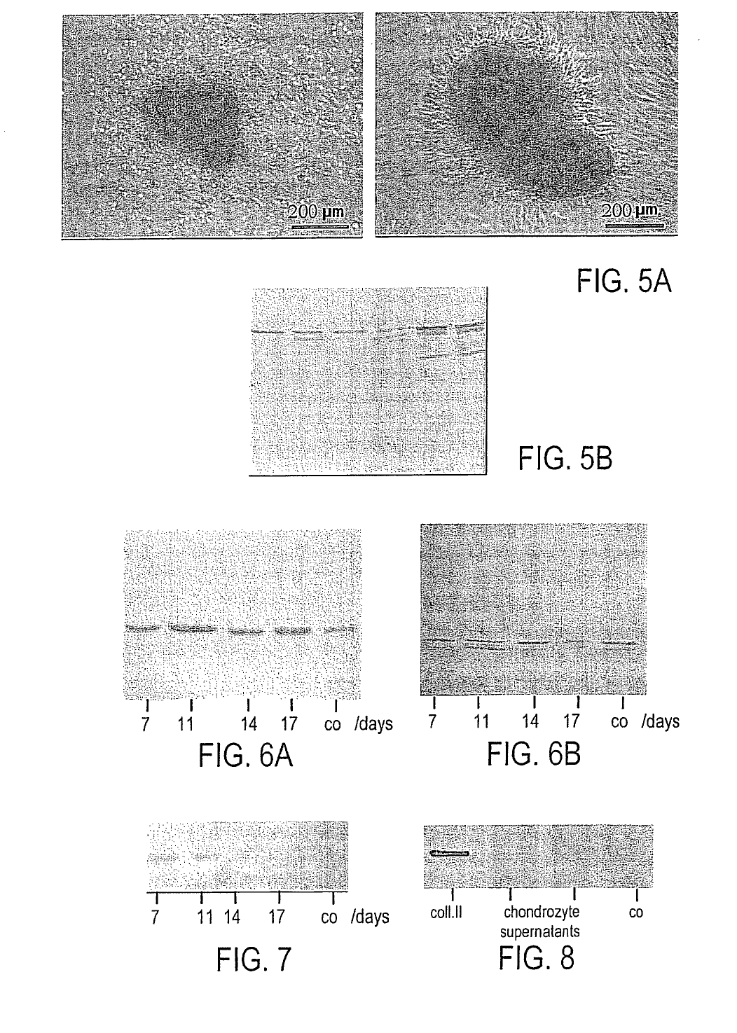

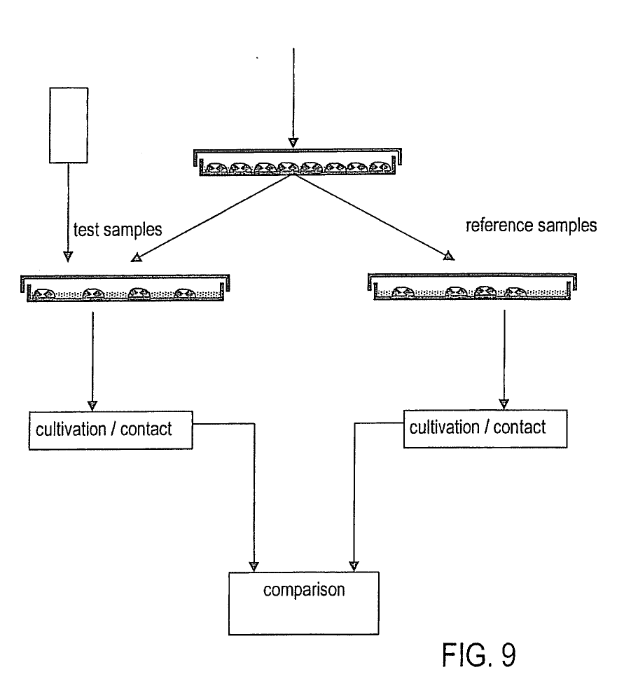

According to the scheme shown in FIG. 1, in order to obtain the stem cells exocrine glandular tissue, e.g., acinar tissue, preferably from a salivary gland or the pancreas, is taken into culture mechanically and enzymatically comminuted (step 10 in FIG. 1). In contrast to the indications of Bachem et al., Gastroenterol. 115:421-432 (1998), and Grosfils et al., Res. Comm. Chem. Pathol. Pharmacol. 79:99-115 (1993), no tissue blocks from which cells are to grow out are cultivated but rather the tissue is more strongly comminuted under the condition that the cell aggregates of the acini remain intact to a very large extent.

These cells and cell aggregates are cultivated in culture vessels for several weeks. Every 2 to 3 days the medium is changed, all differentiated cells being removed at this time. The cells persisting in culture are undifferentiated cells with unlimited capacity to divide.

Similar cells have been isolated under the same conditions from the pancreas and described and designated as a type of myofibroblasts or pancreatic astrocytes (Bachem et al., 1998). However, in contrast to the cells of the present invention an unlimited capacity to divide could not be observed. Furthermore, these cells could also not be passaged in an unlimited manner without losing vitality.

In a second step (12) approximately 400 to 800 cells are cultivated in 20 μl medium each in hanging drops. To this end the drops are placed on the cover of bacteriological Petri dishes, turned over and placed over the Petri dish filled with medium so that the drops hang downward.

As a result of this type of cultivation cell aggregates (14) referred to as organoid bodies form within 48 h, which are transferred into a suspension culture for approximately 6 days (16). The partial view (18) in FIG. 1 shows a micrograph of such an organoid body.

The organoid bodies growing in suspension culture form new organoid bodies that also induce the formation of new organoid bodies in individual cells. The cells can be frozen as organoid bodies as well as individual cells and retain their vitality and their differentiation potential.

FIGS. 2-4 show micrographs and electron micrographs of differentiated cells obtained from such organoid bodies.

For example, the formation of a neuromuscular network could be observed thereby:

Cells obtained from OBs strongly expressed α-SMA (smooth-muscle actin) (FIGS. 2e-f). The presence of wide-spread bundles of myofilaments that extended through the cytoplasm was confirmed by electron microscopy (FIGS. 4a-c). Furthermore, cells were identified that were immunoreactive for the pan-neuron marker PGP 9.5 and for neurofilaments (NF). The neurofilament system extended from the pericaryon into the radial cytoplasmatic processes (FIGS. 2c,d). PGP 9.5-immunoreactive cells displayed numerous varicosities along their branched processes (FIGS. 2a, b, 4d) and thus resembled typical morphological features of autonomous nerve fibers. Cells that were immunoreactive for GFAP (glial fibrillary acidic protein) were in close proximity to cells that expressed neuronal markers (FIGS. 2c, d). The filamentary proteins frequently did not extend through the entire cytoplasm but rather were limited to areas adjacent to the nerve cells. Furthermore, smooth muscle cells and nerve cells were not randomy scattered but rather formed connected networks with junctions that could be readily distinguished (FIGS. 2e,f). Nerve fiber processes extended over considerably large distances in order to contact adjacent smooth muscle cells as their presumed targets. Thus, the two cell types exhibited features of a primitive neuromuscular network based on their topographical arrangement. An incipient formation of tissue-like structures was observed in 3 weeks old OBs (FIGS. 2g-j). Here, a cluster of fibrous nerve cells was found in contact with glia cells (FIG. 2g) or was further developed to a three-dimensional neuromuscular network (FIG. 2h), which was confirmed in cross sections of 8 weeks old OBs (FIG. 2i,j).

Detection of Expression of Exocrine and Endocrine Pancreatic Proteins:

Immunohistochemical stainings have demonstrated that cellular subsets were positive for amylase (FIG. 2k). The immunoreaction signal was limited to clearly distinguishable vesicles within the apical cytoplasm. In addition, most of the cell clusters that were immunoreactive for amylase were arranged in circles, where the secretory vesicles had a position towards the middle, which is a morphological arrangement similar to that of exocrine pancreatic acini. Other cellular subsets showed immunoreactivity for insulin (FIG. 21). Similarly to the amylase-positive cell clusters, the secretory product was stored in vesicular structures that were concentrated on a cell pole. The presence of secretory cells has been confirmed by electron microscopy, which showed densely distributed electrodense particles like those characteristic of excretory or incretory functions (FIGS. 4f-h).

A Differentiation into Chondrogenous Cells and Epithelial Cells was also Observed:

After a growth period of two months OBs displayed chondrogenous properties. An Alcian blue staining revealed areas with high concentrations of proteoglycans (chondroitin sulfate), that occurred either as globular (FIG. 3a) or fibrillary (FIG. 3b) deposits. Immunohistochemical stainings with antibodies directed against the cartilage matrix protein collagen II additionally documented the chondrogenous activity within these globular (FIG. 3c) and fibrillary (FIG. 3d) areas. The immunoreactivity was highest in the middle of the cellular aggregates that most likely corresponded to areas of developing extracellular cartilage matrix. This observation was confirmed by confocal microscopy (FIG. 3g): whereas the amount of collagen depots increased toward the middle of the cellular aggregates, the border areas were characterized by actively translating cells as demonstrated by their high expression of vigilin that is usually found in cells with active translational machinery, e.g., collagen-synthesizing chondrocytes or in fibroblasts during chondroinduction. Typical individual collagen II translating chondrocytes have also observed in out-growing cells of OBs that produced a collagen II-containing matrix surrounding the individual cells (FIG. 3e). An ultrastructure examination of these areas was able to clearly show a network of reticular fibers and collagen fibers, the latter being identified by their characteristic band pattern (FIG. 4e). In addition to mesenchymal markers, a few cells also expressed several cytokeratins, which indicates their potential for differentiation into epithelial cells. However, cells that were immunoreactive for cytokeratins were found less frequently than cells that expressed the markers of smooth muscle cells and neurons. They were typically arranged in clusters disseminated within the OBs (FIG. 3f). Typical cell contacts between keratinocytes were found by electron microscopic examinations (FIG. 4j) and epithelial cells were found on the surface in 8 weeks old OBs which surface grew out of the cell culture medium into the air.

Altogether, e.g. the following markers for specific cells so far could be tested positive: PGP 9.5 and NF for nerve cells, S 100 and GFAP for glia cells, SMA for muscle cells (and/or myofibroblasts), collagen type II for cartilage cells, amylase and trypsin for exocrine glandular cells, insulin for endocrine glandular cells, vigilin for strongly translating cells and cytokeratin for epidermal cells. In addition to the light microscopic examinations, it was possible to characterize different cell types morphologically in electron microscopy and cell-cell contacts were found as a sign for cellular interactions as well.

So far smooth muscle cells, neurons, glia cells, epithelial cells, fat cells, cardiac cells, kidney cells, fibroblasts (e.g., skin- and tendon fibroblasts), chondrocytes, endocrine and exocrine glandular cells and thus cell types of all three germ layers in these organoid bodies, among others, have been demonstrated morphologically/histologically and/or immuno-chemically.

The present invention will be explained in detail in the following non-limiting examples.

The general working instructions customary for methods for cultivating mammalian cells, in particular human cells, are to be observed. A sterile environment in which the method is to be carried out is to be observed in any case, even if no further description for this is given. The following buffers and media were used:

| | |
|---|---|
| HEPES stock | 2.383 g HEPES per 100 ml A. bidest. |
| solution (pH 7.6) | |
| HEPES Eagle's | 90 ml modified Eagle's Medium (MEM) |
| Medium (pH 7.4) | 10 ml HEPES stock solution |
| Isolation | 32 ml HEPES Eagle's Medium |
| medium (pH 7.4) | 8 ml 5% BSA in A. bidest. |
| | 300 μl 0.1 M CaC1₂ |
| | 100 μl trasylol (200,000 KIU) |
| Digestion | 20 ml Isolation medium |
| medium (pH 7.4) | 4 ml collagenase (collagenase NB 8 |
| | from Serva) |
| Incubation medium | Dulbecco's modified Eagle's Medium (DMEM) |
| Nutrient medium | Dulbecco's modified Eagle's Medium (DMEM) |
| | DMEM + 4500 mg/l glucose + |
| | L-glutamine − pyruvate + |
| | 20% PCS (inactivated) + 1 ml/100 ml pen/ |
| | strep (10000 U/10000 μg/ml) |
| | or |
| | DMEM + 10% autoplasma + 1 ml/100 ml |
| | pen/strep, warm to 37° C. before use |
| Differentiation | 380 ml DMEM |
| medium | 95 ml 30 min at 54° C. inactivated PCS |
| | 5 ml glutamine (GIBCO BRL) |
| | 5 ml (3,5 μl β-mercaptoethanol per 5 ml PBS) |
| | 5 ml nonessential amino acids (GIBCO BRL) |
| | 5 ml penicillin/streptomycin (GIBCO BRL) |
| | (10000 U/10000 μg/ml) |

Instead of fetal calf serum (FCS) in the nutrient medium and differentiation medium, plasma or serum of another suitable species, especially human plasma, or less preferably, human serum, may be used as well.

Instead of the DMEM medium used, the nutrient medium can also contain another known base medium suitable for the cultivation of eukaryotic cells, especially mammalian cells, as base medium in which the differentiated cells die and the desired stem cells proliferate. The isolation medium, incubation medium and differentiation medium may also contain a different customary and suitable base medium.

The following examples 1 to 3 describe working protocols for isolating and cultivating adult pluripotent stem cells from acinar tissue of the pancreas or from acinar and tubular tissue of the salivary gland.

### EXAMPLE 1

In order to isolate and cultivate human adult stem cells human tissue was obtained from adult patients immediately after a surgical intervention and prepared at once. Healthy tissue was separated from the surgically removed tissue, e.g., pancreatic tissue, and taken up (at 20° C., lesser metabolism) in digestion medium containing HEPES Eagle's medium (pH 7.4), 0.1 mM HEPES buffer (pH, 7.6), 70% (vol./vol.) modified Eagle's medium, 0.5% (vol./vol.) trasylol (Bayer AG, Leverkusen, Germany), 1% (wt./vol.) bovine serum albumin), 2.4 mM CaCl₂ and collagenase (0.63 P/mg, Serva, Heidelberg, Germany). The pancreatic tissue was very finely comminuted with shears, fatty tissue floating on top removed by suction and the tissue suspension gassed with Carbogen (Messer, Krefeld, Germany) without the nozzle entering into the medium with the cells (reduction of mechanical stress) and adjusted therewith to pH 7.4. The suspension was then incubated in a 25 ml Erlenmeyer flask (covered with aluminum foil) under constant agitation (150-200 cycles per minute) at 37° C. in 10 ml digestion medium. After 15-20 minutes the fat floating on top and the medium were removed by suction and the tissue was again comminuted and rinsed with medium without collagenase (repeat procedure at least twice, preferably until cell fraction transparent), whereupon digestion medium was added and another gassing was performed for approximately 1 minute with Carbogen. A digestion with collagenase followed again for 15 minutes at 37° C. in an agitator using the same buffer. After the digestion the acini were dissociated by successively drawing them up and ejecting through 10 ml, 5 ml and 2 ml glass pipettes with narrow openings and filtered through a single-layer nylon mesh (Polymon PES-200/45, Angst & Pfister AG, Zurich, Switzerland) with a mesh size of approximately 250 μm. The acini were centrifuged (at 37° C. and 600-800 rpm in a Beckman GPR centrifuge, corresponds to approximately 50-100 g) and further purified by being washed in incubation medium containing 24.5 mM HEPES (pH 7.5), 96 mM NaCl, 6 mM KCl, 1 mM MgCl₂, 2.5 mM NaH₂PO₄, 0. mM CaCl₂, 11.5 mM glucose, 5 mM sodium pyruvate, 5 mM sodium glutamate, 5 mM sodium fumarate, 1% (vol./vol.) modified Eagle's Medium, 1% (wt./vol.) bovine serum albumin, equilibrated with Carbogen and adjusted to pH 7.4. The washing procedure (centrifugation, removal by suction, re-suspension) was repeated five times. Unless otherwise indicated, the work was performed at approximately 20° C. in the above isolation.

The acini were re-suspended in incubation medium and cultivated at 37° C. in a moist atmosphere with 5% CO₂. The acinar tissue died rapidly (within two days) and the dying differentiated cells separated from the adjacent cells without damaging them (gentle isolation) and the stem cells that were not dying sank to the bottom, to which they adhered. The differentiated acini cells were not capable of doing this. The incubation medium was replaced for the first time on the second or third day after the seeding, where a large part of the freely floating acini and acinar cells was removed. At this time the first stem cells or their precursors, respectively, had settled on the bottom and began to divide. The medium replacement was repeated thereafter on every third day and differentiated acinar pancreatic cells were removed at each medium replacement.

On the seventh day in culture the cells were passaged with a solution consisting of 2 ml PBS, 1 ml trypsin (+0.05% EDTA) and 2 ml incubation medium, during which the cells separated from the bottom of the culture dish. The cell suspension was centrifuged 5 minutes at approximately 1000 rpm (Beckmann GPR centrifuge), the supernatant removed by suction and the cells re-suspended in 2 ml incubation medium and transferred to a medium-sized cell culture bottle to which 10 ml incubation medium were added.

On the fourteenth day in culture the cells were passaged again but this time with 6 ml PBS, 3 ml trypsin/EDTA and 6 ml incubation medium. The cell suspension was centrifuged 5 minutes at 1000 rpm, the supernatant removed by suction and the cells re-suspended in 6 ml incubation medium, transferred to 3 medium cell culture bottles and 10 ml incubation medium added to each one.

On day 17 a third passaging took place to a total of 6 medium cell culture bottles and on day 24 a fourth passaging to a total of 12 medium cell culture bottles. Now at the latest all primary cells except for the stem cells had been removed from the cell culture.

The stem cells can be cultivated further and passaged and seeded as often as desired. The seeding preferably takes place at a density of 2-4×10⁵ cells/cm² in incubation medium.

### EXAMPLE 2

Pancreatic acini were obtained from male Sprague-Dawley rats (20-300 g) that had been narcotized (CO₂) and exsanguinated via the dorsal aorta. A cannula was introduced transduodenally into the pancreatic duct and 10 ml digestion medium that contained HEPES Eagle's medium (pH 7.4), 0.1 mM HEPES buffer (pH, 7.6), 70% (vol./vol.) Modified Eagle's medium, 0.5% (vol./vol.) trasylol (Bayer AG, Leverkusen, Germany), 1% (wt./vol.) bovine serum albumin, 2.4 mM CaCl₂ and collagenase (0.63 P/mg, Serva, Heidelberg, Germany) injected into the pancreas from the rear.

Prior to the removal the pancreas had been partially freed of the adhering fatty tissue, lymph nodes and blood vessels.

Then, fresh pancreatic tissue was taken into digestion medium (at 20° C., lesser metabolism) and the pancreatic tissue very finely comminuted with shears and processed as described in example 1.

### EXAMPLE 3

The isolation and cultivation from exocrine tissue of the parotid gland took place analogously to the pancreas protocol with the following deviations:

1. The exocrine tissue of the parotid gland was a mixture of acinar tissue and tubular tissue.

2. Since salivary glands contain less proteases and amylases than the pancreas, it is possible to store the salivary gland tissue for a while in a refrigerator at approximately 4° C. without the tissue being damaged too much. In the concrete exemplary case the storage time was 15 h and entailed no disadvantageous consequences for the isolation of the desired stem cells.

The following examples 4 and 5 describe in detail two protocols for producing organoid bodies and differentiated cells.

### EXAMPLE 4

The undifferentiated cells are trypsinated with a solution of 10 ml PBS, 4 ml trypsin, 8 ml differentiation medium and centrifuged off for 5 minutes. The resulting pellet is re-suspended in differentiation medium in such a manner that a dilution of 3000 cells per 100 μl medium is adjusted. The cells are subsequently well suspended again with a 3 ml pipette.

The cover is removed from bacteriological Petri dishes, which had previously been coated with 15 ml PBS (37° C.) per plate, and inverted. Approximately fifty 20 ml drops are placed with the aid of an automatic pipette on a cover. The cover is then rapidly inverted and placed on the Petri dish filled with differentiation medium so that the drops hang downward. The Petri dishes are subsequently carefully placed in an incubator and incubated for 48 h.

Then, the cells that are aggregated in the hanging drops, which cells are to be referred to as organoid bodies (OB) herein, are transferred from four covers at a time into one bacteriological Petri dish with 5 ml incubation medium with 20% FCS and cultivated for another 96 h.

The organoid bodies are now carefully collected with a pipette and transferred into cell culture vessels coated with 0.1% gelatin and containing differentiation medium. In an especially preferred embodiment of the method 6 cm Petri dishes coated with 0.1% gelatin into which 4 ml differentiation medium had been placed and that are subsequently each loaded with 6 organoid bodies are used as culture vessel. Another preferred culture vessel are chamber slides coated with 0.1% gelatin into which 3 ml differentiation medium had been placed and that are subsequently each loaded with 3-8 organoid bodies. In addition, 24-well microtiter plates can also be used that were coated with 0.1% gelatin and into which 1.5 ml differentiation medium had been placed per well and that are subsequently coated with 4 organoid bodies each.

Cultivated in this manner, the differentiation capacity of the cells into the organoid bodies is activated and the cells differentiate into cells of the three germ layers, mesoderm, entoderm and ectoderm. The cells can be stored and cultivated as organoid bodies as well as individual cells and retain their pluripotency.

### EXAMPLE 5

Stem cells after the 42nd day of cultivation were preferably used for the induction of the differentiation. The use of stem cells after the 3rd or 4th passage or of cells that had been stored at the temperature of liquid nitrogen for 12-18 months was also possible without problems.

At first, the cells were transferred into differentiation medium with the composition indicated above and adjusted to a density of approximately 3×10⁴ cells/ml, e.g., by trypsin treatment of a stem cell culture in nutrient medium, 5-minute centrifugation at 1000 rpm and re-suspension of the pellet in differentiation medium and dilution to the extent required.

Subsequently, approximately 50 20-μl drops (600 cells/20 μl) were placed on the inside of the cover of a bacteriological Petri dish (plugged tips) using a 20 μl pipette and the cover was carefully inverted onto the Petri dishes filled with PBS so that the drops hung downward. A new tip was used for each cover. The Petri dishes were subsequently carefully placed into the incubator and incubated 48 h at 37° C.

Then, the aggregated cells in the hanging drops, the organoid bodies (OB), were transferred from four covers at a time into one bacteriological Petri dish with 5 ml incubation medium with 20% FCS (hold cover obliquely and rinse the organoid bodies off with approximately 2.5 ml nutrient medium) and cultivated for another 5-9 days, preferably 96 h.

The organoid bodies were now carefully collected with a pipette and transferred into cell culture vessels coated with 0.1% gelatin and containing differentiation medium. The organoid bodies now multiplied and grew in partially individual cell colonies that were again able to be multiplied, isolated and multiplied. In an especially preferred embodiment of the invention 6 cm Petri dishes coated with 0.1% gelatin were used as culture vessels into which 4 ml differentiation medium had been placed and they were each loaded with 6 organoid bodies. Another preferred culture vessel was chamber slides, coated with 0.1% gelatin into which 3 ml differentiation medium had been placed and that were each subsequently loaded with 3-8 organoid bodies, and Thermanox plates (Nalge Nonc International, USA) for electron microscopic studies. Another alternative was 24-well microtiter plates coated with 0.1% gelatin into each of which 1.5 ml differentiation medium per well had been placed and that were subsequently each loaded with 4 organoid bodies.

In a preferred embodiment of the method organoid bodies were cultivated approximately 7 weeks in the gelatin-coated 6 cm Petri dishes and thereafter individual organoid bodies were cut out with the Microdissector (Eppendorf, Hamburg, Germany) according to the instructions of the manufacturer and then transferred, e.g., onto fresh 6 cm Petri dishes, chamber slides or Thermanox plates. In a further preferred embodiment individual OBs were separated with pipette tips by gentle aspiration and transferred, followed by, e.g., observation under an inverse microscope.

### EXAMPLE 6

### Characterization of Differentiated Cells in the Organoid Bodies

1. Immunohistochemistry

Organoid bodies, that had been cultivated at least 3 weeks on chamber slides, as well as cross sections of “long-time” OBs were rinsed twice in PBS, fixed for five minutes with methanol:acetone (7:3) containing 1 g/ml DAPI (Roche, Switzerland) at −20° C. and washed three times in PBS. After incubation in 10% normal goat serum at room temperature for 15 minutes the samples were incubated overnight with primary antibodies at 4° C. in a moistening chamber. The primary antibodies were directed against the protein gene product 9.5 (PGP 9.5, polyclonal rabbit antibody, 1:400, Ultraclone, Insel Wight), neurofilaments (NF-Pan-Cocktail, polyclonal rabbit antibody, 1:200, Biotrend, Germany), α-smooth muscle actin (α-SMA, monoclonal mouse antibody, 1:100, DAKO, Denmark), glial fibrillary acidic protein (GFAP, monoclonal mouse antibody, 1:100, DAKO, Denmark), collagen II (monoclonal mouse antibody, II-II-6B3, 1:20, Developmental Studies Hybridoma Bank, University of Iowa, USA), vigilin FP3 (1:200, Kügler et al., 1996), cytokeratins (Pan Cytokeratin, monoclonal mouse antibody, 1:100, Sigma, USA), alpha-amylase (polyclonal rabbit antibody, 1:100, Calbiochem, Germany) and insulin (monoclonal mouse antibody, 0.5 g/ml, Dianova, Germany). After having been rinsed three times with PBS, slides were incubated 45 minutes at 37° C. with either Cy3-marked anti-mouse IgG or FITC-marked anti-rabbit IgG (Dianova), both diluted 1:200. The slides were washed three times in PBS, coated with Vectashield Mounting Medium (Vector, USA) and analyzed with a fluorescence microscope (Axiosop Zeiss, Germany) or with a confocal laser scanning microscope (LSM 510 Zeiss, Germany). An Alcian blue staining was performed with standard methods.

2. Transmission electron microscopy

OBs were cultivated 3 weeks on Thermanox plates (Nalge Nonc International, USA). Samples adhering to the Thermanox plates were incubated at pH 7.4 for 24 h by being immersed in 0.1 M cacodylate buffer containing 2.5% glutaraldehyde and 2% paraformaldehyde. After a post-fixing in 1% OsO₄, “en bloc” staining with 2% uranylacetate and dehydration in pure alcohols the samples were embedded in Araldite. After removal of the Thermanox plate, semithin cuts were performed either tangentially or verticalloy to the embedded cell culture and stained with methylene blue and azure II. Ultrathin sections were cut out of the regions of interest, stained with lead citrate and examined under a transmission electron microscope (Phillips, EM 109).

The following examples 7 to 10 describe the contacting of organoid bodies, that were produced as described above, with different active substances and the determination of a change, caused by the particular active substance, in the organoid bodies and/or in the differentiated cells contained in them by direct visual detection or the detection of marker proteins.

### EXAMPLE 7

In this example and in the following ones several jointly multiplied organoid bodies of a batch (e.g., by separating suitable organoids and renewed enlargement until the desired number is produced), usually at least 6 in a group, are used for a test.

In this example organoid bodies were exposed for a time period of 1 to 2 days to different micromolar concentrations of puromycin, an active substance that inhibits translation. Then, the size of the treated organoid bodies was compared with those of an untreated control (see comparative micrograph in FIG. 5A). In a second detection method the organoids were taken up in 10 ml lysis buffer containing 7.5 ml PBS, 2.5 ml NP-40 and 1 mM PEFA block, stored overnight in a refrigerator at 4° C. and then homogenized in minihomogenizers for Eppendorf tubes. The homogenate was centrifuged, the supernatant removed and resolved electrophoretically according to standard methods and the gel subjected to a Western immunoblot. FIG. 5b shows the results of the treatment via the detection of the translation marker vigilin. The first four bands are produced by the different active amounts of puromycin, the last two show the amount of vigilin in the untreated organoids; the same amount of total protein was always applied per track.

### EXAMPLE 8

The organoid bodies were incubated 7, 11, 14 and 17 days with 2×10⁻⁶ M retinoic acid or without retinoic acid. Then, the organoid bodies were homogenized as described in example 7 and subjected to a Western blot assay. The markers α-SMA (α-smooth muscle actin) and a mixture of neurofilaments (NF) were stained (FIG. 6). Whereas the amount of actin is higher during the entire treatment time than in the control, the amount of synthesized NF detectable in the Western blot changes only on the 7^{th} and the 11^{th} day of treatment.

### EXAMPLE 9

The organoid bodies were incubated with 40 ng/ml HGF (hepatocyte growth factor) or without HFG for 7, 11 14 and 17 days. Then, the organoid bodies were homogenized as described in example 7 and subjected to a Western blot assay. The production of the liver protein α-fetoprotein was examined (FIG. 7). After 7 and 11 days of incubation a distinct synthesis of the fetoprotein can be observed whereas a longer exposure time tends to inhibit the production again.

### EXAMPLE 10

Organoid bodies were incubated without or with conditioned medium of chondrocytes primary cultures and the presence of collagen II as typical cartilage protein was examined again with a Western blot as above (FIG. 8). Both batches with chondrocyte culture supernatants showed a slight increase of collagen II compared with the control with simple medium.

The above examples only demonstrate a basic procedure in the carrying out of the present invention with a small number of chemical active substances and detection methods. However, the invention is in no way limited to these exemplary embodiments. Alternatives, especially alternative detection methods, are known to those skilled in the art or can be readily found using the detailed disclosure of this application in combination with the cited state of the art.

The features of the invention disclosed in the present description, the claims and the drawings can be significant both individually as well as in combination for realizing the invention in its different embodiments.

1-30. (canceled)

31. A method for testing substances regarding their effect on biological cells using organoid bodies formed by aggregation and differentiation of multipotent or pluripotent adult stem cells, comprising:
bringing the substance to be tested into contact with the organoid bodies; and
determining an effect of said contact, if any, by a detectable change in said organoid bodies and/or in the cell types contained therein.

32. The method according to claim 31, wherein said substance to be tested is any one selected from the group consisting of a known or potential active substance or a mutagen.

33. The method according to claim 31, wherein said substance to be tested is any one selected from the group consisting of active drug substances and cosmetics.

34. The method according to claim 31, wherein the cell aggregates are mammalian cell aggregates.

35. The method according to claim 34, wherein said mammalian cell aggregates are human cell aggregates.

36. The method according to claim 31, wherein the substance to be tested is at least one selected from the group consisting of a protein, peptide, a nucleic acid, DNA, RNA, a derivative thereof, and a low-molecular weight chemical substance.

37. The method according to claim 31, wherein said organoid bodies were formed by aggregation and differentiation of multipotent or pluripotent adult stem cells isolated from exocrine glandular tissue.

38. The method according to claim 37, wherein the exocrine glandular tissue is an acinar tissue.

39. The method according to claim 31, wherein the effect of said contact on said organoid bodies is at least one selected from the group consisting of a change in morphology, a change in capacity for proliferation, a change in capacity for growth, a change in viability, a change in an activity of all cell types of said organoid bodies, and a change in an activity of specific cell types of the organoid bodies.

40. The method according to claim 31, wherein determining an effect of said contact by a detectable change in said organoid bodies comprises detecting the presence or absence of a marker substance produced by the organoid bodies.

41. The method of claim 40, wherein said marker substance is produced by at least some cell types of the organoid bodies.

42. The method of claim 40, wherein detecting the presence or absence of said marker substance further comprises detecting an amount of said marker substance.

43. The method according to claim 40, wherein said marker substance is a marker protein.

44. The method according to claim 43, wherein said the marker protein is detected by at least one selected from the group consisting of the binding of dyes, antibodies or receptors, a Western blot, and enzymatic or other activity of the marker protein.

45. The method according to claim 40, wherein said marker substance is at least one selected from the group consisting of a nucleic acid, DNA, RNA and any derivative thereof.

46. The method according to claim 31, wherein determining the effect of the substance comprises use of at least one method selected from the group consisting of protein assays, immunoassays, enzymatic assays, receptor binding assays, ELISA assays, RIA assays, electrophoretic and chromatographic assays, HPLC, Northern blots, Southern blots, Western blots, calorimetric assays, immunohistochemical, electrophysiological, microscopic and spectroscopic detection.

47. The method according to claim 31, wherein the cell types of the organoid bodies are selected from the group consisting of osteoblasts, osteoclasts, chondrocytes, adipocytes, fibroblasts, muscle cells, endothelial cells, epithelial cells, hematopoietic cells, sensory cells, endocrine and exocrine glandular cells, glia cells, neuronal cells, oligodendrocytes, blood cells, intestinal cells, cardiac-, lung-, liver-, kidney- and pancreatic cells.

48. The method according to claim 47, wherein the cell-type composition of the organoid bodies was determined by cultivation in a medium containing cell-type-specific differentiation factors and/or growth factors.

49. The method according to claim 48, wherein the growth factors and/or differentiation factors are selected from the group consisting of bFGF, VEGF, DMSO and isoproterenol, fibroblast growth factor 4 (FGF4), hepatocyte growth factor (HGF), TGF beta1, EGF, KGF, retinoic acid, beta-NGF, BMP-4 and activin-A.

50. The method according to claim 31, wherein two or more of the different cell types of the organoid bodies form structures that are similar to tissues or organs.

51. The method according to claim 50, wherein two or more of the different cell types of the organoid bodies form neuromuscular structures or glia-nerve cell structures or skin cell structures.

52. The method according to claim 50, wherein the substance brings about a change in said structures.

53. The method according to claim 52, wherein the change is at least one selected from the group consisting of destruction of the structures, inhibition or stimulation of the development of the structures and a change in the activity of one or more cell types from which said structures are composed.

54. The method according to claim 31, wherein the organoid bodies are located in at least one consisting of hollow spaces, matrices, carrier systems and shaping systems.

55. The method according to claim 31, comprising bringing two or more different substances into contact with said organoid bodies.

56. The method according to claim 55, comprising simultaneously bringing said two or more different substances into contact with said organoid bodies.

57. The method according to claim 55, comprising successively bringing said two or more different substances into contact with said organoid bodies.

58. A device for carrying out the method for testing substances regarding their effect on organoid bodies, comprising:
organoid bodies formed by aggregation and differentiation of multipotent or pluripotent adult stem cells;
means for contacting the organoid bodies with a substance to be tested; and
means for detecting a change in the organoid bodies or in cell types contained therein.

59. The device of claim 58, further comprising at least one selected from the group consisting of a suitable container, carrier system or shaping system for said organoid bodies

60. A kit for carrying out a method for testing substances regarding their effect on organoid bodies, comprising organoid bodies formed by aggregation and differentiation of multipotent or pluripotent adult stem cells in a suitable culture medium for maintaining the organoid bodies.

61. The kit according to claim 60, wherein the organoid bodies are located in at least one consisting of hollow spaces, matrices carrier systems, and shaping systems.

62. The kit according to claim 60, further comprising means for contacting the organoid bodies with a substance to be tested.

63. The kit according to claim 60, further comprising means for detecting a change in said organoid bodies or in the cell types contained therein.

64. The kit according to claim 63, further comprising other reagents and auxiliary substances.

65. A kit for carrying out a method for testing substances regarding their effect on organoid bodies, comprising:
multipotent or pluripotent adult stem cells suitable for producing organoid bodies, said adult stem cells being in a suitable culture medium for maintaining said adult stein cells; and
reagents to detect a change in the organoid bodies or in cell types contained therein.

66. The kit according to claim 65, further comprising differentiation factors and culture media to produce organoid bodies with a desired cell type composition.

67. The kit according to claim 65, further comprising means for contacting the organoid bodies with a substance to be tested.

68. The kit according to claim 65, further comprising means for detecting a change in said organoid bodies or in the cell types contained therein.
